(19) United States
(12) Patent Application Publication
(10) Pub. No.: US 2005/0186256 A1
(43) Pub. Date: Aug. 25, 2005

(54) Dissolvable film comprising an active ingredient and method of manufacture

(76) Inventors: Deborah L. Dihel, Whitehouse Station, NJ (US); Ghislaine Joly, Bridgewater, NJ (US)

Correspondence Address: Cynthia L. Foulke;NATIONAL STARCH AND CHEMICAL COMPNAY, 10 Finderne Avenue, Bridgewater, NJ, 08807-0500, US

(21) Appl. No.: 10/783,078

(22) Filed: Feb. 20, 2004

(51) Int. Cl. A61K 9/16; A61K 9/50

(52) U.S. Cl. 424/443; 264/4

(57) ABSTRACT

A film having disposed thereon a substance for delivery to a predetermined substrate. The substance is encapsulated in a water soluble encapsulant. The film disintegrates when place on the surface of certain substrates where it releases the substance to the local environment. The films can carry medicaments, flavors, seasonings, scents and the like.

### FIELD OF THE INVENTION

The invention relates to films comprising an active ingredient. The active ingredient may desirably be encapsulated in order to preserve the active during film forming.

### BACKGROUND OF THE INVENTION

Film compositions that exhibit instant wettability followed by rapid dissolution have been used to deliver or administer therapeutic or cosmetic substances, food flavor-imparting agents including food flavorings, or other ingredient contained within the film. See, e.g., U.S. Pat. Nos. 6,596,298, 6,419,903, and 6,231,957. Such films generally comprise a water-soluble edible polymer such as, for example, pullulan and/or starch. Upon exposure to an aqueous environment, e.g., the oral cavity, the film dissolves whereby the substance contained therein is released.

Volatile flavors, however, are easily lost during processing of the film or during storage of the film. While flavors and other actives can be stabilized to protect against oxidation and volatilization by encapsulating them in water soluble or oil soluble capsules, water soluble capsules that are added to the film pre-mix, will quickly dissolve and release their flavors prior or during film formation, and oil soluble capsules may melt during the heat exposure of the process, or if they survive, will not be released into the mouth quickly to impart a flavor burst.

There is thus a need in the art for a method of preparing films that can be used for the delivery of a substance, in particular a volatile substance, in order to expand the types of substances that can be delivered using a dissolvable film. The current invention addresses this need.

### SUMMARY OF THE INVENTION

It has been discovered that when a substance is applied to a film after film coating, but just prior to drying, the film will be sticky enough to adhere the substance. The substance to be adhered can be a substance, the size of which is too large for incorporation into the film itself, or, can be an encapsulated substance. Encapsulation in a water-soluble matrix enables the administration, via the film delivery method, of volatile substances.

When a substance that has been encapsulated within a water soluble matrix is applied to the film after film coating, but just prior to drying, the film will be sticky enough to adhere the encapsulated substance, yet the exposure to a high moisture environment limited enough not to dissolve the encapsulating matrix. The result is a dried water soluble film containing a substance (i.e., the active ingredient) in a water soluble encapsulant.

The present invention provides a film that can be used to deliver a substance, such as a flavor or a therapeutically active ingredient, and is particularly useful when the substance to be delivered is a volatile substance.

One aspect of the invention provides a film having disposed on at least one surface thereof a substance. In a preferred embodiment, the active substance is an encapsulated substance. In a particularly preferred embodiment, the encapsulated substance is a volatile substance. Preferred films for use in the practice of the invention comprise pullulan and/or starch, more preferable the starch is a modified starch. Hydroxyalkylated starch and succinated starch are particularly preferred modified starches for use in the practice of the invention.

Another aspect of the invention provides a method of making a film comprising a substance. The method comprises mixing film-forming ingredients together to form a mixture, coating the mixture onto a suitable substrate to form a film, applying a substance to the film, and then drying the film to a moisture content of less than about 15 weight % moisture, preferable from about 5 weight % to about 15 weight % moisture, even more preferably from about 6 weight % to about 10 weight % moisture. The formed film having the substance applied thereon can be air-dried or dried under warm air. The film may then be cut to the desired dimension, packaged and stored. In one embodiment the substance applied to the film is an encapsulated substance.

Yet another aspect of the invention is directed to a method of delivering a desired substance to a desired substrate, upon which delivery the desired substance is released. In the method of the invention the desired substance is present on the surface of a film, and the film is delivered to the desired substrate.

Still another aspect of the invention is directed to a method of preserving the flavor of a substance to be delivered by a film. The method comprises encapsulating the substance in a water soluble encapsulant and placing the encapsulated substance on the surface of the film. In a preferred embodiment the encapsulated active is placed on the film after film forming stage, but prior to drying stage.

### DETAILED DESCRIPTION OF THE INVENTION

The present invention relates to a film that can be used to administer a substance, such as flavors, in particular volatile substances. Substances that are too large, such as spices e.g., parsley flakes or the like, can be sprinkled onto the surface of the film. Volatile substance that would be substantially lost during film manufacture can be incorporated into a water soluble encapsulant and delivered using the film and manufacturing techniques of the invention.

The terms “substance” and the term “active” are used interchangeably herein to refer to the ingredient intended for delivery, i.e., the film serves as the carrier or vehicle for delivery of the ingredient.

The term “substantially aqueous environment” means the environment wherein the carrier film rapidly dissolves, releasing the active. Typically, the substantially aqueous environment will be within oral cavity, e.g., the surface on the tongue, or may be a food product such as a glass of water or juice, soup or the like.

The term rapidly dissolves means that the carrier film dissolves in less than about 60 seconds.

It is to be understood that both the film and active may dissolve in the aqueous environment, or the film, encapsulating material and active can dissolve, or only the film and encapsulating material dissolves and a non-soluble active released into the aqueous environment, after which it may be e.g., swallowed.

In film making, flavors added to the film slurry before being coated have a tendency to volatilize and disappear over time, leaving only a sweet or savory film with no real identity. This is particularly noticeable when using water-soluble flavors or flavors having low flash point flavors. While a flavor can be protected by encapsulating it, if the encapsulating agent is hydrophilic, like starch encapsulation, the encapsulation will not survive the process of mixing and/or cooking in a water medium before being coated.

It has been discovered that use of a soluble encapsulation is possible by sprinkling the encapsulated flavor onto a film after coating the film but before drying the film. It has been found that the encapsulated flavor has not enough residency time/shear to solubilize and will remain encapsulated upon drying of the film. It has also been found that sprinkling or dusting the film with encapsulated flavor after the drier may also be used to preserve the flavor.

Film-Forming Composition

The film-forming composition used in the practice of the invention is not particularly limiting. The composition should be strong, flexible, be blocking and moisture resistant so that it does not adhere to itself, yet be able to dissolve quickly when placed in a substantially aqueous environment.

Water soluble solid film-forming agents conventionally used in the dissolvable film-forming art can be used in the current invention. Such water soluble polymers include, but are not limited to pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and various mixtures thereof.

The film may be made by a variety of processes known in the art. For example, the starch may be dispersed with the other film components in water or other solvent and dried into film form. In the alternative, the starch and other dry components may be blended and then dispersed with any additional film components in water or other solvent and dried into film form. Films may be formed from such dispersions or solutions by shaping it into a solidified form of a suitable thickness by any technique known in the art including, but not limited to, wet casting, freeze-drying, and extrusion molding. The dispersion or solution may also be directly coated or sprayed onto another edible product, such as a tablet or foodstuff, and dried to form an edible film.

A particularly suitable process for preparing the films of the present invention is by preparing a coating formulation by making a solution or dispersion of the film components, applying the mixture to a substrate, using knife, bar or extrusion die coating methods, drying the coated substrate to remove the majority of the solvent, and removing the film from the substrate. Suitable substrates include, but are not limited to, silicone elastomers, metal foils and metalized polyfoils, composite foils or films containing polyetrafluoroethylene materials or equivalents thereof, polyether block amide copolymers, polyurethanes, polyvinylidene chloride, nylon, polyethylene, polyester, and other such materials useful in the art as releasable substrates.

The film is not completely dried in that some degree of water or other solvent remains. The amount of water may be controlled to obtain desired functionality. For example, more water typically results in a more flexible film, while too much water results in a film that will block and be tacky.

The film thickness will depend, in part, on the desired end use. Typically, the film thickness will be in the range of about 1 to 500 microns, particularly 25 to 100 microns. When prepared as an oral film for quick dissolution in the oral cavity, the film thickness is more preferably from about 25 to 50 microns. Thicker films may be use in e.g., food preparation applications or the like.

The films exhibit moisture and blocking resistance, yet are quickly wetted when exposed to water, such as when placed on the tongue or other substrate surface, followed by rapid dissolution and/or disintegration. The wettability and dissolution rates of the starches may be modified by one skilled in the art to target a specific delivery profile. For example, more rapid dissolution is typically preferred when the film is an oral film and particularly suitable films for such use are those which completely dissolve in less than about 30 seconds, particularly less than about 20 seconds, more particularly in less than about 10 seconds. For other uses, less rapid dissolution is necessary and films may completely dissolve in no more than about 60 seconds, particularly less than about 45 seconds, more particularly less than about 30 seconds.

One skilled in the art can also modify the film formulation to provide clarity and other desired characteristics by manipulation of the pullulan and/or starch component and control of other components.

The films may be used for delivering any active agent for a variety of applications including personal care, skin care, wound care, pharmaceutical, and breath freshening. For example, the film may be used to deliver antiseptic, a drug, or aromatizing agent to the mouth, i.e. to treat bad breadth, cold sore or the like. In addition, the film may be used for delivery actives such as flavorings to tea, coffee or the like or for delivering spices to soups, marinades or the like, for delivering oils to bath water. It will be appreciated that the foregoing represents a non-exhaustive list of end use applications.

The films may also be used to provide a fragrance, such as lavender oil for use in, e.g., aromotherapy in bath or potpourri pots.

In addition to human applications, veterinary, agricultural and horticultural applications are contemplated.

The flavorings that can be used include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lernon); ethyl vanillin (vanilla, cream); heliotropine, i.e., piperonal (vanilla, cream); vanillin (vanilla, cream); alpha-amyl cinnarnaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 26-dimethyloctanal (green fruit); and 2-dodecenal (citrus, mandarin); cherry; grape; mixtures thereof; and the like.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.01 to about 10 wt % are useable.

Pullulan is a natural neutral polysaccharide, repeatedly polymerized by maltotriose (three alpha 1,4 linkaged glucose) via alpha-1,6 linkages. It is a white power that is tasteless, odorless, amorphous and non-crystalline. Pullulan is prepared by fermenting a starch hydrolyzate with the yeast Aureobasidium pullulan, filtering to remove cellular material, purifying, concentrating, drying and pulverizing. Transparent films can be made from an aqueous solution of pullulan.

Starch, as used herein, is intended to include all starches derived from any native source, any of which may be suitable for the films of the invention. A native starch, as this term is used herein, is one as it is found in nature. Also suitable are starches derived from a plant obtained by standard breeding techniques including crossbreeding, translocation, inversion, transformation or any other method of gene or chromosome engineering to include variations thereof. In addition, starch derived from a plant grown from artificial mutations and variations of the above generic composition, which may be produced by known standard methods of mutation breeding, are also suitable.

Typical sources for the starches include cereals, tubers, roots, legumes and fruits. The native source can be corn, pea, potato, sweet potato, banana, barley, wheat, rice, sago, amaranth, tapioca, arrowroot, canna, sorghum, and waxy or high amylose varieties thereof. A “waxy” starch is defined as a starch containing at least about 95% by weight amylopectin. A “high amylose” starch is defined as a starch containing at least about 40% by weight amylose.

Preferred starch-based films will comprise a modified starch. Preferably, at least about 50%, more preferably at least about 65%, and even more preferably at least about 90% of the starch will be a modified starch. The starch may be modified using any modification technique known in the art, including physical, chemical and/or enzymatic modifications.

Physically modified starches, such as sheared starches, or thermally-inhibited starches described in the family of patents represented by WO 95/04082, may be suitable for use herein.

Chemically modified products are also intended to be included as the base material and include, without limitation, those which have been crosslinked, acetylated and organically esterified, hydroxyethylated and hydroxypropylated, phosphorylated and inorganically esterified, cationic, anionic, nonionic, and zwitterionic, and succinate and substituted succinate derivatives thereof. Such modifications are known in the art, for example in Modified Starches: Properties and Uses, Ed. Wurzburg, CRC Press, Inc., Florida (1986).

Conversion products derived from any of the starches, including fluidity or thin-boiling starches prepared by oxidation, enzyme conversion, acid hydrolysis, heat and or acid dextrinization, thermal and or sheared products may also be useful herein.

Further suitable are pregelatinized starches which are known in the art and disclosed for example in U.S. Pat. Nos. 4,465,702, 5,037,929, 5,131,953, and 5,149,799. Conventional procedures for pregelatinizing starch are also known to those skilled in the art and described for example in Chapter XXII-“Production and Use of Pregelatinized Starch”, Starch: Chemistry and Technology, Vol. III Industrial Aspects, R. L. Whistler and E. F. Paschall, Editors, Academic Press, New York 1967.

Any starch or starch blend having suitable properties, for use herein may be purified by any method known in the art to remove starch off flavors and colors that are native to the polysaccharide or created during processing. Suitable purification processes for treating starches are disclosed in the family of patents represented by EP 554 818 (Kasica, et al.). Alkali washing techniques, for starches intended for use in either granular or pregelatinized form, are also useful and described in the family of patents represented by U.S. Pat. No. 4,477,480 (Seidel) and U.S. Pat No. 5,187,272 (Bertalan et al.).

Particularly suitable starches are starches capable of emulsifying or encapsulating the active ingredient so that there is no need for additional encapsulating or emulsifying agents, Such starches include, without limitation, hydroxyalkylated starches such as hydroxypropylated or hydroxyethylated starches, and succinated starches such as octenylsuccinated or dodecylsuccinated starches. The use of emulsifying or encapsulating starches are particularly useful in that a solution or dispersion of the film material (starch component, active agent, and optional additives) may be stored for later processing. The hydroxyalkylated starches have the added advantage of forming a softer film so that there is less or no need for a plasticizer.

To facilitate processing of the films, the starches are typically at least partially converted to reduce the viscosity and allow for the production of a high solids starch dispersion/solution, such as 30% solids starch dispersion/solution. Particularly suitable starches are those with a viscosity of at least about 1000 cps at 20% solids and a viscosity of no more than about 10,000 cps at 90% solids.

Particularly suitable starches have a flow viscosity of at least about 7 seconds, more particularly at least about 10 seconds and no more than about 19 seconds, particularly no more than about 15 seconds. Flow viscosity, as used herein, is measured by the test defined in the Examples section, below.

The molecular weight of the starch is also important to its functionality in a film, particularly to film strength. For example, dextrins are not suitable in the present application.

The starch component may be a single modified starch, a blend of modified starches, or a blend of modified and native starches. Blends may be particularly useful to lower the cost of the film or to more easily achieve a variety of desirable properties and functionalities. If native starches are used, they may only be used in minor amounts, particularly less than 15%, more particularly less than about 10% by weight of the starch component.

The starch component may also comprise a cellulosic material or a gum, such as pullulan which is fully compatible and essentially substitutable for the starch. Other cellulosic materials and gums include without limitation carboxymethyl cellulose, hydroxypropyl cellulose, microcrystalline cellulose, ethylcellulose, cellulose acetate phthalate, hydrocolloids, carageenan, gums, and alginate. However, a cellulosic material or a gum is not an essential component of the film and may be used at levels of less than about 15 percent, more particularly less than about 10 percent by weight of the starch component, or may even be absent from the film. As starch is generally less expensive than pullulan, the cost of a pullulan film may be decreased by substituting starch for at least a portion of the pullulan, particularly at least about 50%, more particularly at least about 85%, most particularly at least about 90% of the pullulan by weight, without loss of the essential functionality of the pullulan film.

At least one plasticizer may be added to increase the apparent flexibility of the films. Further, a solid polyol plasticizer will generally provide better resistance to moisture absorption and blocking. One skilled in the art can chose a plasticizer to meet the desired needs of the film, such as choosing an edible plasticizer for an oral film. Plasticizers useful in the instant invention include, polyols, polycarboxylic acids, and polyesters. Examples of useful polyols include, but are not limited to ethylene glycol, propylene glycol, sugar alcohols such as sorbitol, manitol, maltitol, lactitol; mono-, di- and oligosaccharides such as fructose, glucose, sucrose, maltose, lactose, and high fructose corn syrup solids and ascorbic acid. Examples of polycarboxylic acids include, but are not limited to, citric acid, maleic acid, succinic acid, polyacrylic acid, and polymaleic acid. Examples of polyesters include but are not limited to glycerin triacetate, acetylated-monoglyceride, diethyl phthalate, triethyl citrate, tributyl citrate, acetyl triethyl citrate, acetyltributyl citrate. The plasticizer may be present in any desired amount, particularly from 0 to about 25 percent, more particularly from about 2 to about 10 by weight of the starch component.

Optional components may be added for a variety of reasons including without limitation, sweeteners, both natural and artificial; emulsifiers; humectants; surfactants; colorants; proteins such as gelatins; gums; flavors and flavor enhancers. Such optional components are typically added in minor amounts, particularly less than about 30% total by weight based upon the weight of the starch component.

The following examples are is presented for purpose of illustration only.

### EXAMPLES

### Example 1

This example describes adding an encapsulated favor after film drying as a dusting agent.

An edible dissolvable film used for a confectionary product was prepared by mixing the components shown in Table 1 in a Waring blender until well blended. The mixture was heating in a 1100 W microwave for 1 minute, high power. The solution was allowed to cool to room temperature and coated using a BRAIVE LABORATORY BENCHTOP COATER, NR434002024 on a PET coating material. The coating gap was adjusted to achieve a dry film thickness of between about 30 microns. The film was dried in a forced air oven at 140° F. for approximately 10 minutes.

TABLE 1

| Film forming components | % |
|---|---|
| Modified starch | 21.00 |
| Acesulfame K | 0.30 |
| Aspartame | 0.30 |
| Sorbitol | 1.33 |
| Polysorbate 80 | 0.05 |
| color | little |
| Carrageenan | 0.17 |
| CHERRY VANILLA (QUEST) | 5.00 |
| glycerol | 4.85 |
| water | 67.00 |
| Total | 100.00 |

The resulting film was peeled off the carrier web and cut into pieces of a suitable size and shape.

A portion of the film samples were subsequently dusted with an encapsulated flavor (I.e. Black Cherry Flavor WONF product code QL38544).

The powdered flavor was encapsulated in a water soluble shell, such as a combination of modified food starch or gums (I.e., gum acacia) and maltodextrins.

Encapsulation prevents volatilization of the flavor and reduces oxidation and extends shelf like. The shelf life of the encapsulated flavor used in this example is 12 months.

The film samples were dusted by gently mixing the film pieces with the encapsulated flavor by tumbling them together in a plastic bag or by applying the encapsulated flavor to the film with a dry paint brush. The encapsulated flavor adhered to the film and excess was shaken off. The films were cut to size and were then packaged in suitable containers such as plastic film vials with front opening.

### Example 2

This example describes adding an encapsulated favor before film drying.

An edible dissolvable film used for a confectionary product was prepared as described in Example 1.

Immediately after forming the film, the encapsulated flavor was added by sprinkling the powdered flavor on top of the wet film. The powdered film adhered to the wet film.

The film was dried in a forced air oven at 140° F. for approximately 10 minutes depending on thickness.

The resulting film was peeled off the carrier web and cut into pieces of suitable size and shape. The films were then packaged.

### Example 3

Films were prepared as described in Examples 1 and 2 except that the flavor used was not encapsulated (Control A and Control B, respectively). The intensity and character of the flavor was determined. Results are shown in Table 2.

TABLE 2

| Sample | Intensity | Character |
|---|---|---|
| Example 1 | High | Cherry |
| Control A | Low | Cherry |
| Example 2 | High | Cherry |
| Control B | Low | Cherry |

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of making a dissolvable film comprising a substance, the method comprising mixing film forming ingredients together to form a mixture, coating the mixture onto a suitable substrate to form a film, applying a substance to the film, and drying the film to a moisture content of less than about 10 weight % moisture.

2. The method of claim 1 wherein the substance is an encapsulated substance.

3. The method of claim 2 comprising a water soluble encapsulant.

4. The method of claim 1 wherein the formed film having the substance applied thereon is air-dried.

5. The method of claim 1 wherein the formed film having the substance applied thereon is dried under warm air.

6. A dissolvable film having disposed on at least one surface thereof an encapsulated substance.

7. The film of claim 6 the encapsulated substance is a volatile substance.

8. The film of claim 6 wherein the encapsulated substance is a flavor.

9. The film of claim 6 comprising pullulan and/or starch.

10. The film of claim 9 comprising a modified a modified starch.

11. The film of claim 10 wherein the modified starch is hydroxyalkylated starch or succinated starch.

12. A method of preserving the flavor of an active to be delivered by a film, the method comprising encapsulating the active in a water soluble encapsulant and placing the encapsulated active on the surface of the film.

13. The method of claim 12 wherein the encapsulated active is placed on the film after film forming stage, but prior to drying stage.
